(19) United States
(12) Patent Application Publication
(10) Pub. No.: US 2025/0154579 A1
(43) Pub. Date: May 15, 2025

(54) SYSTEM AND METHOD FOR HIGH THROUGHPUT SCREENING OF SMALL MOLECULE-PROTEIN INTERACTIONS

(71) Applicant: INTERPLAY BIO, Ann Arbor, MI (US)

(72) Inventor: Pavan Kommareddi, Ypsilanti, MI (US)

(73) Assignee: INTERPLAY BIO, Ann Arbor, MI (US)

(21) Appl. No.: 18/388,850

(22) Filed: Nov. 12, 2023

(51) Int. Cl. C12Q 1/6869; C12N 15/10

(52) U.S. Cl. CPC C12Q 1/6869; C12N 15/1037; C12N 15/1096

(57) ABSTRACT

A system and method for the high throughput screening of small molecule-protein interactions includes first immobilizing selected small molecules on a solid matrix. Immobilization is preferably made by photo affinity labelling in which a linker used to bind the small molecule to the matrix. A cDNA library is established from a broad array of human tissues. The tissue mRNA is converted to cDNA and transferred to a phage genome to make a phage display library which is mixed with small molecules coupled to the beads and incubated. The incubation mixture is washed to remove unbound phage including non-specific and weak binders. The bound phage are amplified and are used in the next round of biopanning. The biopanning cycles are repeated followed by PCR of individual plaques selected from the final biopanning round. The PCR products are barcoded from each small molecule experiment and are pooled followed by Nextgen sequencing.

### TECHNICAL FIELD

The disclosed invention relates generally to the identification of small molecule-protein interactions. More particularly, the disclosed invention relates to a system and method for the high throughput screening of small molecule-protein interactions.

### BACKGROUND OF THE INVENTION

In the pharmaceutical industry, an increasing amount of attention is being given to new uses for known pharmaceuticals. During drug development, it is common for the drug researchers to target specific diseases. Little consideration is understandably given at the time of development to alternative or potential applications of the drug to other disease states, particularly those that are not related to the disease initially being targeted. As a consequence, investment of both time and resources into drug discovery fall short of realizing the true potential of the discovered drug.

Efforts have been made to find other uses for known drugs in an effort to expand the application of the drug to its fullest. These methodologies typically involve the screening of protein-protein binding interactions. While providing useful advances in alternative or additional drug application discovery, known screening methods are burdened by overly complex protocols. According to the most common approach, a bacteriophage display library is first established after which the protein-small molecule interactions are identified and evaluated once immobilization of the small molecule takes place. However, this technique relies on altering the chemistry of each molecule under investigation, thus resulting in a cumbersome and slow process.

Accordingly, it is desirable to provide a method in which the interactions of small molecules can be quickly identified and matched to one or more diseases linked with the identified protein. The desired method would provide for the high throughput immobilization of small molecules to a solid matrix followed by rapid identification of relevant interactions.

### SUMMARY OF THE INVENTION

In devising a solution to accelerating flow through in the analysis of interactions of small molecules, a particularly useful technique is photo-crosslinking of small molecules to a solid surface which provides a valuable tool to elucidate protein small molecule interactions. This technique helps to identify binding partners for drug discovery as well as in chemical biology research.

According to the disclosed invention, selected small molecules are first immobilized on a solid matrix which may be any one of a variety of materials having a solid surface and being useful to this purpose, including but not limited to styrene, beads, particularly though not exclusively silica beads, and magnetic beads having aminated surfaces, provided that there is an amine on the surface. Immobilization—the process of attaching or tethering biomolecules such as proteins to a solid surface—is preferably made by photo affinity labelling. Particularly, a linker, which is preferably a photo crosslinker such as but not limited to the group diazirines, is used to bind the small molecule to the matrix.

Along with the immobilization of the small molecules, a cDNA library is established from a broad array of human tissues. The tissue mRNA is converted to cDNA and transferred to a phage genome to make a phage display library.

Once the cDNA library is established, the phage library is mixed with small molecules coupled to the beads and incubated for different time points based on the particular experiment. The incubation mixture is washed to remove unbound phage. The washing is done thoroughly to remove non-specific and weak binders. The bound phage are amplified by infecting them with E. coli. The amplified phage are used in the next round of biopanning. The biopanning cycles are preferably repeated between four and eight times followed by PCR of individual plaques selected from the final biopanning round. The PCR products are barcoded from each small molecule experiment. The barcoded PCR products are pooled and are then subjected to Nextgen sequencing. The pooling of products allows for a significant improvement in the number of small molecules which can be screened for interactions in a single next generation sequencing compared with known approaches.

The resulting protein-small molecule interactions which are identified following screening are then correlated with relevant literature to identify any known disease pathways involved with the protein. The protein-small molecule interactions are validated based on their hypothesis testing result.

Other advantages and features of the embodiments of the invention will become apparent when viewed in light of the detailed description of the preferred embodiment when taken in conjunction with the attached drawings and the appended claims.

### BRIEF DESCRIPTION OF THE DRAWINGS

For a more complete understanding of this invention, reference should now be made to the embodiment illustrated in greater detail in the accompanying drawings and described below by way of examples of the invention wherein:

FIG. 1 is a flow chart illustrating the steps provided for the high throughput screening of small molecule-protein interactions according to the present invention;

FIG. 2 is a schematic illustrating the NHS-diazirine crosslinking reaction in which the small molecules are linked to the solid matrix; and

FIG. 3 illustrates the key steps involved in the panning cycle.

### DETAILED DESCRIPTION OF THE PREFERRED EMBODIMENT

In the following description, various operating parameters and components are described for one constructed embodiment. These specific parameters and components are included as examples and are not meant to be limiting.

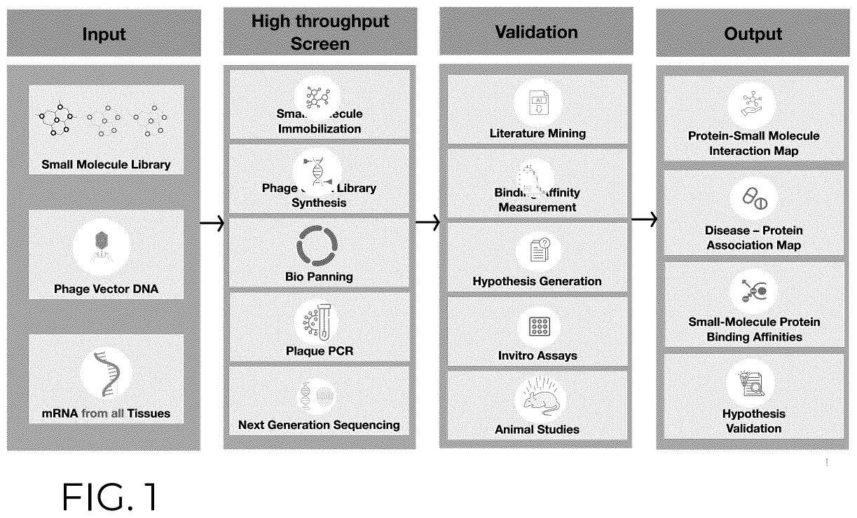

With reference to FIG. 1, a perspective view of a flow chart illustrating the steps provided for the method of high throughput screening of small molecule-protein interactions according to the present invention. The method includes four essential procedures, with each essential procedure including a subset of steps.

The four essential procedures include Input, High Throughput Screening, Validation, and Output. Of the four essential procedures, three—Input, Validation, and Output—are familiar to those skilled in the art.

The first essential procedure, the Input step, includes the steps of selecting an established small molecule library (or creating a small molecule library) which includes one or more of the small molecules under study. The library is not necessarily fixed and can be adapted as needed to include a number of small molecules. The selection of a bacteriophage vector DNA is made to identify the appropriate phage vector. Preferably though not absolutely the bacteriophage T7 (or the “T7 phage”) is the preferred bacteriophage for use in the present method. While a number of bacteriophages may be suitable for the intended purpose set forth herein, the T7 is preferred as it is overall an excellent cloning vector. The T7 phage is regarded as a well-characterized, double-stranded DNA bacteriophages which is relatively easy to grow. The T7 phage particle is very vigorous and replicates quickly. uniquely able to survive harsh conditions, the T7 phage offers superior survivability over other phages, thereby withstanding environments which are known to inactivate phage which do not offer the same level of robustness.

The last step of the Input step is the extraction of mRNA from the selected human tissue. While human tissue may be the tissue of choice as a general rule, animal tissue may also or alternatively be chosen.

The second essential procedure, the High Throughput Screening, includes four sub steps, that of small molecule immobilization, phage cDNA library synthesis, plaque PCR, and Next Generation Sequencing.

Once the small molecule of interest for crosslinking is selected, the small molecule is coupled to a bead having a linker with diazirine, by exposing the small molecule-beads mixture to UV 350 nm from 15 minutes to 60 minutes. The diazirine (azipentanoate) moiety has better photostability than phenyl azide groups, and it is more easily and efficiently activated with long-wave UV light (330 to 370 nm). According to the diazirine reaction scheme for a light-activated photochemical reaction, one end of a crosslinker has the diazirine group and the other end is the NHS or COOH used to link to an amine group on solid surface being linked. Ultimately, irradiation of diazirines with ultraviolet light leads to formation of highly reactive carbene species, which are inserted into nearby C—H, N—H, and O—H bonds of small molecules.

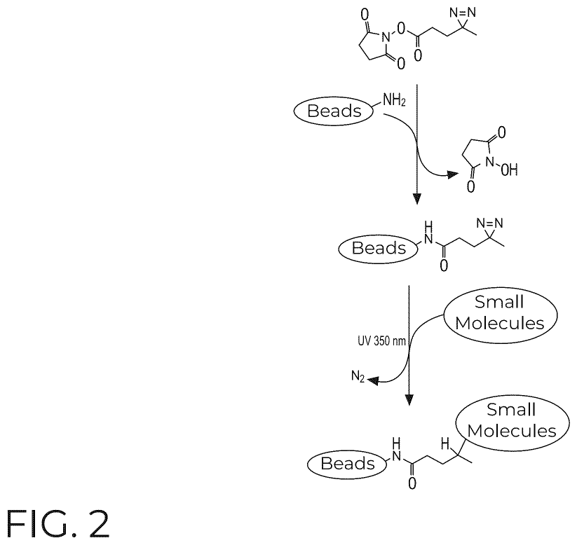

The selection of the appropriate matrix is important to the positive outcome of the study. Common choices include the above-mentioned styrene, silica beads, magnetic beads, but may also include glass slides, or gold nanoparticles, surface plasmon resonance chips, and Bio-layer interferometry probes in addition to other surface structures. Immobilization of the small molecule on the matrix is accomplished through cross-linking, such as by photocrosslinking. According to the present invention, the photocrosslinker binds to a beads as illustrated in FIG. 2 in which the NHS-diazirine crosslinker is attached to aminated surfaces on beads through NHS. The diazirine is now free to react with small molecules. The small molecule to be immobilized is added to the linker attached beads and is then exposed to UV light (350 nm). This UV exposure leads to the generation of highly reactive carbene species that react with nearby C—H, N—H, and O—H bonds of small molecules covalently.

A cDNA phage library is synthesized in which the preferred phage, in this case T7 is preferably though not absolutely used. The cDNA phage library may be formed from a broad variety of preferred bacteriophages. The members of the phage libraries each carry a different protein.

The exposure to UV light forms a very active carbonyl moiety which reacts with all of the CH groups. Accordingly, the reaction is broad and generally the binding reaction takes place with more than one group. For example, binding can occur with CH groups, NH groups, and OH groups among others. At this stage orientation can be a problem as all molecules include CH groups, thus orientation can occur in any direction. The result is a high throughput of interactions under study. This approach compares very favorably to known techniques in which the chemistry of the molecule is changed, resulting in a very slow and costly process. Through the procedure of immobilization set forth in the disclosed invention, throughput is increased dramatically.

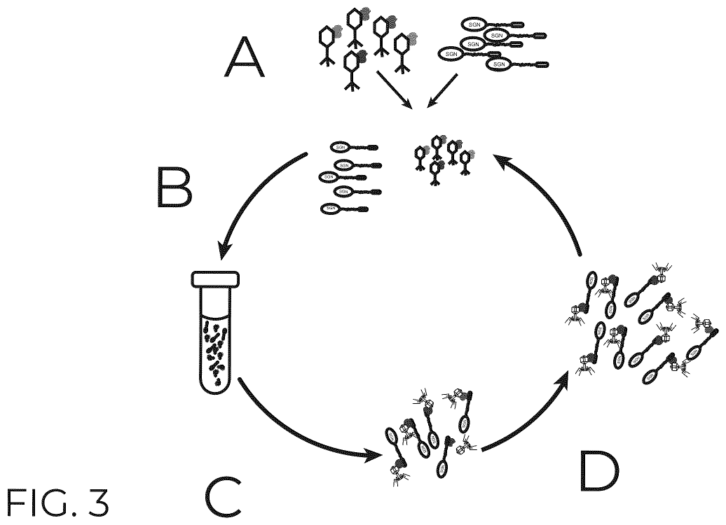

A panning cycle is illustrated in FIG. 3. In general, and with reference to FIG. 3, “A” illustrates the step of making a phage cDNA library, “B” illustrates the step of incubating the mixture of the phage library with selected small molecules coupled to the matrix, “C” illustrates the step of washing and eluting the incubated mixture to remove unbound phage, and “D” illustrates the step of amplifying the unbound phage.

During the panning cycle, the phage is introduced. Any protein that binds to this small molecule will be held in place and the balance will be washed off through the process of biopanning, a step which is repeated multiple times, preferably between 4 and 8 times. The molecules without specific binding sites can only randomly bind, thus their amplification is limited. However, the molecules with specific binding sites, the actual binders, will exponentially amplify compared with non-specific molecules because these molecules do not possess a binding site. Washing is repeated to remove the non-binding molecules.

The amplification step is undertaken by PCR which, at present is done manually, but is under development for automation. Following amplification and biopanning, the sample is plated on a lawn of bacteria. Regardless of where the phage is positioned, there will be lysis of the bacteria called plaques. The T7 phage plaques resulting from biopanning are subjected to PCR. This method allows identification of the specific molecules involved in binding proteins.

Once biopanning is complete, the phage plaques are sequenced to determine which protein is expressed from their coat. The practical impact of the disclosed invention is the benefit of the significant throughput.

The sub step of sequencing follows and is itself an improvement over known techniques in which individual phage are subject to PCR. According to the present invention, a large mix of sequences in the range of between 200-1000s may be accomplished at the same time in a single tube. The plaque PCR products from each small molecule screening experiment are bar coded and multiple experiments can be mixed (up to 96, the number of wells in a standard well dish) and NGS sequencing done to reduce the cost of sequencing as well as increasing the coverage of screened phage. Each experiment is assigned a single bar code, so in this instance there will be 96 bar codes.

The third essential procedure, the Validation step, includes different methods which may be suitable only when the initial study results are known. The object of validation is to confirm that binding has in fact occurred and to validate it through a different method, such as, surface plasmon resonance, Bio layer interferometry, thermal shift assay etc. Followed by researching known literature to serve as potential instruction as to the results of the studies. Some literature may only be discoverable once the protein-molecule interactions are known following execution of the method. However, only testing of the cell line will confirm whether or not the actual desired binding has occurred.

The fourth essential procedure, the Output step, depends on mapping, both protein-small molecule interaction mapping and, importantly for the purpose of the method of the present invention, disease-protein association mapping. Affinities for small molecule-protein binding are reviewed and confirmed during the Output step. Finally, the hypothesis is validated, and the results are used to establish the basis for pharmaceutical development.

The foregoing discussion discloses and describes an exemplary embodiment of the present invention. One skilled in the art will readily recognize from such discussion, and from the accompanying drawings and claims that various changes, modifications and variations can be made therein without departing from the true spirit and fair scope of the invention.

I claim:

1. A method for high throughput screening of small molecule-protein interactions comprising:
selecting a tissue;
selecting small molecules for screening;
forming a matrix;
immobilizing the selected molecules on a matrix;
converting the tissue mRNA to cDNA;
establishing a cDNA library;
transferring the cDNA to a phage genome to make a phage display library;
mixing the phage library with the selected small molecules coupled to the matrix;
incubating the mixture for different time points based on the particular experiment;
washing the incubated mixture to remove unbound phage as non-specific and weak binders;
amplifying the bound phage;
biopanning using the amplified bound phage;
repeating biopanning;
undertaking PCR of the individual plaques selected from the final biopanning round;
barcoding the PCR products from each small molecule experiment;
pooling the barcoded PCR products;
subjecting the pooled PCR products to sequencing;
correlating the resulting protein-small molecule interactions identified following screening with literature to identify disease pathways involved with the protein; and
validating the protein-small molecule interactions.

2. The method of claim 1, wherein the matrix is formed from styrene, silica beads, glass, and magnetic beads.

3. The method of claim 1, wherein the magnetic beads have aminated surfaces.

4. The method of claim 2, wherein the magnetic beads are oligo d(T25) magnetic beads.

5. The method of claim 1, wherein immobilization is made by photo affinity labeling.

6. The method of claim 1, wherein a linker is used to bind the small molecule to the matrix.

7. The method of claim 6, wherein the linker is a photocrosslinker.

8. The method of claim 7, wherein the photocrosslinker is a diazirine.

9. The method of claim 1, wherein amplification of the bound phage is achieved by infecting them with E. coli.

10. The method of claim 1, wherein sequencing is Nextgen sequencing.

11. The method of claim 1, wherein conversion of the mRNA to cDNA is through the use of directional cloning random primer and oligo dT primer.

12. The method of claim 1, wherein the tissue is selected from the group consisting of animal and human tissue.

13. The method of claim 1, including subjecting the cDNA ends to flushing and phosphorylating.

14. The method of claim 1, wherein the linkers are directional EcoRI/HindIII linkers.

15. The method of high throughput screening according to claim 14, wherein the EcoRI and HindIII sites on the ligated linkers are enzymatically digested.

16. A method for high throughput screening of small molecule-protein interactions comprising:
selecting a tissue;
selecting small molecules for screening;
forming a matrix;
binding the small molecule to a matrix using a linker resulting in immobilization of the small molecule;
converting the tissue mRNA to cDNA;
establishing a cDNA library;
making a phage display library;
mixing the phage library with the selected small molecules coupled to the matrix;
incubating the mixture;
washing the incubated mixture to remove unbound phage;
amplifying the bound phage;
biopanning using the bound phage;
repeating biopanning;
undertaking PCR of the individual plaques selected from the final biopanning round;
barcoding the PCR products;
pooling the barcoded PCR products;
subjecting the pooled PCR products to Nextgen sequencing;
correlating the resulting protein-small molecule interactions identified following screening with literature to identify disease pathways involved with the protein; and
validating the protein-small molecule interactions.

17. The method of claim 16, wherein the linker is a photocrosslinker.

18. The method of claim 17, wherein the photocrosslinker is a diazirine.

19. The method of claim 16, wherein conversion of the mRNA to cDNA is through the use of directional cloning random primer and oligo dT primer.

20. A method for high throughput screening of small molecule-protein interactions comprising:
selecting a tissue;
selecting small molecules for screening;
forming a matrix;
immobilizing the small molecule on the matrix;
converting the tissue mRNA to cDNA;
establishing a cDNA library;
making a phage display library;
mixing the phage library with the immobilized small molecules;
incubating the mixture;
washing the incubated mixture to remove unbound phage;
amplifying the bound phage;
biopanning using the bound phage;
repeating biopanning;
undertaking PCR of the individual plaques selected from the final biopanning round;
barcoding the PCR products;
pooling the barcoded PCR products; and
sequencing the pooled PCR products.
